(19) United States
(12) Patent Application Publication
(10) Pub. No.: US 2009/0202434 A1
(43) Pub. Date: Aug. 13, 2009

(54) Pharmaceutical Composition and Dressing for Treating Skin Lesion, as Well as the Use of Cerium Salt Associated With a Collagen Matrix

(76) Inventor: Luis Eduardo da Cruz, Rio de Janeiro-RJ (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/202,599

(22) Filed: Sep. 2, 2008

(30) Foreign Application Priority Data
Feb. 8, 2008 (BR) PI0800085-9

(51) Int. Cl. A61K 51/00; A61P 17/02

(52) U.S. Cl. 424/1.61

(57) ABSTRACT

The present invention refers to a pharmaceutical composition for treating skin lesion, comprising a cerium salt on a collagen matrix and a dermatologically acceptable carrier. The present invention also refers to a wound dressing for treating skin lesion, comprising the pharmaceutical composition of the present invention. The present invention further refers to the use of a cerium salt associated with collagen in the preparation of the pharmaceutical composition or wound dressing according to the present invention. The composition of the present invention can be used in topical applications in a variety of lesion types, such as skin lesions involving the release of toxins related to microbial proteins on human or animal organisms, or those appointed as HSP; burns which involve burned skin toxin formation or LPC; chronically ulcerate skin lesions in which there is an overproduction of proteinase; skin lesions of difficult resolution, in which control of exudate overproduction is required; and critically infected or colonized skin lesions.

### BACKGROUND OF THE INVENTION

In contemporary medical practice, wound healing treatment is based on the use of dressings, base illnesses control, debridement of non-viable tissue, homeostasis, restoration of adequate tissue perfusion, pressure limitation over wounded spot and infection control.

From a historical point of view, many things have changed on wound treatment approach until present concepts are reached. In ancient Egypt, a wound was seen as a hole through which evil beings coming from hell could enter a person's body. Interpreting things like this, excrement would be applied over the lesion hoping this could send even the worst of demons away. One of the most popular drugs used in ancient Egypt was honey. Nowadays, its therapeutic properties are attributed to repressing microorganism growth and, for the fact of being hygroscopic, to attracting leukocyte and antibodies to the wounded spot.

As far as wound dressings are concerned, the Egyptian used to apply a technology similar to that used in the mummies embalming process. Bandages were used to cover and keep medicine in the desired body spots. Lynen produced in Egypt would vary in texture from fine gauze-like fibers used nowadays to thicker fabric as the ones used with mummies. For lesion debridement there are descriptions of treatment with larvae, able to develop proteolytic enzymes which degenerate the necrotic tissue and liquefy it.

Hypocrites used to recommend cleaning the wounds with warm water, wine and vinegar and drying it. The concept that the wound should be kept dry to provide better healing conditions persisted until the end of the II World War. From then radical changes regarding the basic concepts of wound healing took place. In 1958, Odland saw that the bottom part of a blister would heal faster if its surface was not removed. Later, using lesions of a domesticated pig as a model, Winter showed a faster epithelium repair after occlusion, thus revolutionizing the approach towards wound care. Then came 1963 and Hinman et al. established the beneficial effect of wound occlusion in human beings.

Presently, the wide range of wound healers aiming at not only keeping the spot humid as well as other actions, such as antimicrobial properties, are available in the market.

### SUMMARY OF THE INVENTION

The present invention refers to obtaining a cell proliferation matrix, in the form of a dressing, through tissue bioengineering techniques, as well as its clinical applications, which generally correspond the treatment given to lesions where cutis integrity loss is seen, including skin mucous ulcers of different etiologies, acting as haemostatic, topical healing, antimicrobial and immune modulator.

The highlight of the composition of the present invention is the association of cerium, a metal from the lanthanide series, to a collagen matrix.

All process stages conform to Manufacturing and Control Good Practice procedures as required by the national and international regulatory agencies.

### Theoretical Basis

### Basic Wound Repair Concepts

Far beyond the linear concept triggered by growth factor processes over inflammatory cells, repair represents interaction amongst soluble mediators, extracellular matrix and parenchyma cells. The extracellular matrix molecules can provide signals to genetic expression through integrin receptors and tissue cells interaction with the matrix can change the phenotypes as well as cell functions.

Tissue trauma is followed by a series of events which can be studied divided in phases (homeostasis, inflammatory, tissue formation and wound remodeling). However, these are not mutually exclusive allowing for temporal superposition.

Tissue aggression and the consequent burst of blood vessels will trigger a first sequence of events that will culminate in coagulation, or clotting. The blood clot formed is useful to keep homeostasis, besides providing the provisory matrix for cell migration.

Platelets adhere to interstitial connective tissue and later aggregate to each other. In this aggregation process they release several mediators and express clotting factors. Fibrin clot and thrombin formed on the spot act as a nest for adhesion and aggregation of additional platelets. Platelets fibrinogen, once converted into fibrin by the thrombin, contributes to the fibrin clot.

Platelets have to be considered at this moment, not only for their important role in the making of the homeostatic cover as well as by releasing the cytokines and growth factors exemplified in the Platelets Derived Growth Factor (PDGF) and the Transforming Growth Factor α and β (TGF α and TGF β).

Besides that, the clotting cascade itself, complement compounds and damaged cells, generate a number of chemotactic factors which when in association attract leukocytes to the damaged spot. Endothelial activation by chemotactic stimulates also the endothelial release of elastase and collagenase molecules which, in turn, ease cellular penetration through blood vessels basal membranes.

Leukocyte will perform the cleaning of strange bodies and bacteria found in the system. Their persistence in the place can extend the inflammatory stage and difficult normal repair. On the other hand, generating chemotactic agents of the wound is generally reduced as it is kept “clean”. The neutrophil residue will gradually be expelled with the scab or phagocytized by macrophages or fibroblasts.

Responding to specific chemotactic factors, such as elastin, fibronectin and collagen fragments and TGF β, peripheral blood monocyte continue to be recruited by the wound where they are activated and show a macrophage phenotype. These cells, as well as the platelets start granulation tissue formation. Macrophages are able to debride the tissue, digesting pathogenic organisms, tissue debris and worn out neutrophils. Macrophage seem to perform a fundamental role in the transition between inflammation and repair since they secret fibroblasts growing factors needed to start and spread the tissue remodeling in wounds.

Some hours after the aggression, keratinocytes of the epithelium residual frames move across the wound. Important phenotype changes are observed in the epithelium cells as retraction of the intercellular tonofilaments, dissolution of the intercellular desmosome in their majority, forming of peripheral cytoplasm actin filaments and loss of firm links between dermis and epidermis, which allows epidermis cells to display lateral motility.

Until two days after aggression, the epithelium cells on the edge of the wound start to migrate. Keratinocytes migrating over the wound do not run randomly over a provisory matrix area but really “spare” viable tissue from non-viable tissue. This migration route is mediated by the integrins expressed by the epidermis cells in their membranes, as for example, the keratinocytes do not express receptors to fibrinogen, fibrin, denatured collagen or fibronectin. Thus, migratory epidermis cells avoid clot rich in fibrin/fibronectin and migrate over collagen type I. In the end, keratinocytes migration brings scab discard.

Simultaneously to the re-ephitalialization, proteins from the basal membrane appear again from the edges of the wound to the center. Epidermis cells return to their normal phenotype, firmly sticking to the basal membrane through hemidesmosome and to the neodermis through collagen type VII fibrils.

After approximately four days of aggression, granulating tissue starts to form. It got its name from the granulated appearance seen when it is incised, due to the presence of many newly-formed capillaries.

Angiogenesis is, in a few words, a process mediated by four related phenomena: change in cellular phenotype, induced migration by chemostatic, mitogen stimulation and the appropriate extracellular matrix.

Besides vascular proliferation, fibroplasia is a marked element in the granulation tissue. Platelets and macrophage release a series of cytokine with proliferative and migratory activities to fibroblasts. Later, the fibroblasts themselves will produce cytokine and respond to them in an autocrine way.

For the fibroblasts to migrate an active proteolysis system able to cleave a way for the migration is necessary. Various enzymes derived from fibroblasts, together with the plasmin coming from the serum, seem to perform this role. Those include plasminogen activator, interstitial colagenase (matrix metalloproteinase 1, MMP-1), gelatinase (MMP-2) and estromelisin (MMP-3), as well as serum-derivated plasminogen.

Matrix Metalloproteinases (MMP) are a family of extracellular proteinases responsible for regulating physiological events, not only the remodeling of the extracellular matrix but also influencing other cellular activities, as proliferation and apoptosis. Their action is fine tuned through tissue inhibitors of metalloproteinases (TIMP) and growth factors. Chemotactic factor PDGF, for instance, stimulates the release of these enzymes by the fibroblasts while TGF induces secretion of proteinase inhibitors, in a display of detailed control of extracellular matrix degradation during fibroblasts migration.

Fibroblasts, as macrophages and newly-formed blood vessels, cleave the fibrin clot as they migrate to the wounded spot and lay a new provisory matrix made of hyaluronan and fibronectin. The extracellular matrix, in turn, affects the fibroblasts in their functions of synthesis and re-shape of the matrix itself, interaction known as dynamic reciprocity.

The following stage is marked by the production of a collagen matrix. Summarizing, during skin repair, connective tissue matrix dismissal happens in a sequence set of fibronectin, collagen type III and, later, collagen type I. Production of the latter coincides with an enhancement of wound resistance. Collagen type V will also be enhanced during the granulation tissue development in parallel to the vascularization of the tissue. Besides providing structural support for new tissue resistance, collagen acts on matrix-immersed cells, for example, changing their cellular phenotype or working as a chemotactic element.

After the collagen matrix dismissal, the fibroblasts reshape it and provoke wound contraction. These cells take the phenotype of smooth muscle cells known as myofibroblasts which, through a link to the extracellular matrix (fibronectin and collagen) and to each other, lead to connective tissue compression and wound contraction. Transmission of traction forces depend basically on fibroblasts connection to collagen matrix via the integrin receptors and crossed links among individual collagen bundles.

Transition from a granulation tissue rich in fibroblasts to a relatively acellular matrix are followed from the cellular point of view by fibroblast apoptosis around the tenth day of repair. Capillae regression happens from one to two days after removal of the angiogenesis stimuli, made through another apoptosis via.

Wound remodeling stage is marked by the extracellular matrix remodeling and cellular differentiation or apoptosis. The composition and extracellular matrix granulation tissue structure is a function of time interval and distance between the edges of the wound, that is, on larger wounds, extracellular matrix remodeling and maturation of neo-epidermis, fibroplasias and neovascularization start from the edge of the wound while granulation tissue formation continues to move towards the most central part of the lesion. This makes the extracellular matrix of the wound edges differ from the central extracellular matrix regarding both the qualitative and the quantitative approaches.

The first cell types to undergo apoptosis are the endothelium cells, with a reduction in the capillae number. It will later happen to myofibroblasts and macrophages leading to more acellular wound repair. The extracellular matrix goes on modifying itself along the following months and years, though slowly.

### Some Influencing Factors on Skin Wound Repair

Many factors, both local and systemic, can influence the tissue wound healing process creating unfavorable outcomes, as hypertrophic scars or keloids or even chronic ulcers, as leg ulcers, pressure ulcers and perforating plantar wounds.

It is well know, for example, that due to the richness of the skin annexes, facial lesions will be repaired faster than lesions on the feet. Low temperatures or blood flow deficiencies can also compromise the wound repair process.

Additionally, other local factors as anoxia, abnormal pH, necrosis, infection, hematoma and strange bodies can compromise tissue repair. Hypoxia favors tissue migration and angiogenesis while compromising cell proliferation, collagen synthesis and resistance against bacteria.

On wounds where the repair evolves naturally there is a balance between the MMPs and TIMPs expressions; on the other hand, on chronic ulcerate lesions, as inferior ulcers, there is growing evidence of a local proteolysis increase. Hart et al. highlight that the destructive action of these high levels of proteases can be a compromising factor to wound healing in chronic ulcers. The excessive activity of proteinase seem to deprive wounds of having an initial matrix that could work as a lead to cell migration and framework for matrix storage and growth factor keeping, key elements involved in orchestrating the new tissue making process. A high proteases activity level can even cause damage to cellular surface proteins as growth factors receptors and integrins receptors, damage enough to create an impact on the activity of all cells engaged on the tissue repair process.

Infection is an important cause of repair delay. Although almost all skin wounds are contaminated by the existing flora, pathogenic organisms need to represent a value over the 100.000 bacteria per gram of tissue mark if the clinic infection is to happen. If bacterial colonization is evaluated as critical, it can provoke a longer than desired inflammatory stage and thus compromise repair and if so, should be treated with topical antiseptics.

In optimized wound repair timelines, one should also drain any hematoma and avoid drugs that ease their formation, as anti-clotting and anti-platelet agents. Strange bodies represent a fitting place for bacteria adherence, reducing oxygen tension and wound pH, so they must be removed.

Regarding systemic factors, both the nutritional features and the quality of life (smoking, alcohol abuse), the use of some drugs (as corticosteroids) or systemic diseases, as diabetes mellitus, can compromise the progress of wound repair processes. Old-age patients show a reduction in their protein synthesis, delayed lymphocitary migration and a persisting inflammatory stage, besides being subject to malnutrition risks, concomitant diseases and use of medicament.

The fluid seen in chronic ulcers, as pressure ulcers, venous stasis ulcers and diabetic foot, all inhibit cellular proliferation, mainly of fibroblasts. This fluid is rich in tumor necrosis factor (TNF α). It is supposed that substances able to reduce the level of this cytokine, such as cerium nitrate, can positively module chronic ulcers repair.

Lastly, apart from what was thought in the past, there is presently clear evidence that a dried wound will not heal as well as one treated on a humid environment. The benefits of a humid environment include the promotion of re-ephitalialization, dermis repair and angiogenesis. Topical medicine and occlusive dressings provide a humid environment that helps in the wound repair.

### Collagen

Collagens are glycoprotein of extracellular matrix composed of three chains and forming triple helix along part of their primary sequence. There are 18 types of appointed collagen, from I to XVIII according to the date they were discovered.

The majority of the studies made over the content of collagen found in repairing wounds and artificially induced granulation tissue (implanted sponges) has examined collagen types I and III, since these two collagen have been characterized for some time and their supramolecular structures are clearly established.

Hard helical collagen macromolecules aggregated in fibril sheaves gradually give repair tissue an enhancement in tensile strength and firmness. Besides working to support structure for the new tissue resistance, collagen can have a profound effect on matrix-immersed cells. Peptides derived from collagen, for instance, work as chemotactic for in vitro fibroblasts and can have a similar in vivo effect. Furthermore, intact collagen can change the phenotype and function of a variety of different. These effects can be partially measured through the activation of integrin receptors for collagen α1β1 and α2β1.

Collagen re-shape during the transition period of granulation tissue for a mature repair is dependent on both the continuous synthesis of the collagen as well as on the collagen catabolism. Collagen degradation on wound is controlled by a variety of collagenase enzymes of granulocytes, macrophages, epidermis cells and fibroblasts. These activities are controlled by various similar inhibitors known as tissue inhibitors of metalloproteinases (TIMP), which are then regulated during development and seemingly during wound repair. Cytokines as the TGF β, PDGF and IL-1 and the extracellular matrix itself can perform an important role in modulating collagenase and TIMP in vivo expression.

Wounds gain only about 20 percent of their final Power on the third week, during which fibrillar collagen accumulated relatively fast and was systematically re-shaped by wound contraction driven by the myfibroblasts. In fact, gradual gain in tension power is less related to new collagen dismissal than to additional collagen re-shape, thicker formation of collagen sheaves and a change to cross molecule links. So, the wounded tissue does not have the same resistance than the non-wounded skin. In its maximum resistance output, a scar will have the maximum of 70 percent of the power of the intact skin.

Hart et al. showed that dressings made from collagen are capable of absorbing a wide range of factors present in chronic ulcers and able of making the healing process more difficult, as proteases, free radicals, and ferric ions. Besides that, they have also reported dressings made from collagen are able to attach to one another and protect growth factors like PDGF, maintaining their biological activity and making the environment even more favorable to skin repair.

In ulcers, applied collagen will work as haemostatic, chemotactic and as a matrix for cell migration. Further, it can get linked and inactivate matrix metalloproteinase present in excess in chronic ulcers, and in these conditions, harmful to the tissue repair process. On the other hand, growth factors will be kept in contact with the wound bed and protected from action of proteases. The fact it is bio-compatible is yet another positive point favoring the use of the collagen matrix.

### Cerium Nitrate

The metal cerium holds powerful anti-microbial action and presents low toxicity towards mammal cells. Burkes & McCleskey have shown that cerium salts are toxic in vitro to bacteria and fungi. In 39 bacteria species studied, cerium nitrate inhibited the growth in concentrations in the order of 0.0004 M. Although the biochemical levels in which cerium exerts its bacteriostatic effects are still unknown, the possibilities are many. A change in bacteria cell walls negative charge has been reported, leading to the migration and agglutination of microorganisms. Lanthanide also responds to nucleic acid and makes insoluble complexes.

It has been shown that human burns when in contact with cerium salts for weeks were less frequently colonized by Gram negative bacteria. Fox et al. (1977) reported the association of cerium nitrate and silver sulfadiazine resulting in an increase in clinic efficacy for patients with severe burns. Observations have confirmed topical cerium reverted T lymphocyte failure common to burns. This beneficial effect is related to cerium link to a lipoprotein complex (LPC) existent on burn lesion.

Tumor necrosis factor (TNF-α) is the most powerful inflammatory cytokine. It is well known that excessive release of cytokines has a harmful action over the immunological function. Deveci et al. demonstrated that treating lesions to cerium nitrate (CN) resulted in an increase of interleucin-6 and in a reduction of TNF-α, limiting the extent of the inflammatory reaction. There are then evidences that this metal is useful in treating chronic lesions, by the unorganized presence of mediators of the inflammatory response, like interleukins and TNF14, 31.

Within the context of chronic ulcers and burns, the advantages of adding cerium nitrate to a dressing are: antimicrobial action, immuno modulating action, anti-inflammatory action by reducing levels of TNF-α.

### DESCRIPTION OF THE INVENTION

The invention refers to the production of cellular proliferation matrix, in the form of a dressing, obtained through bioengineering techniques, comprising bovine collagen in association with a cerium salt and, as an option, an alginate with haemostatic, wound repairing, antimicrobial, immunomodulating properties, able to absorb exudates excess, keeping the environment humid, though at the same time preventing maceration of the lesion and whose therapeutic action is related to treating the infection and wound colonization, as a result of its wide spectrum of antimicrobial action.

Its wound healing properties come from collagen acting as a structural support and facilitator of cell migration besides performing a protective role for the newly-produced collagen in a collagenase-rich environment, common in chronic ulcers. Additionally, the presence of toxins on the burn-affected skin, like LPC, and that of inflammatory cytokines largely produced in the environment of chronic ulcers, as Tumor Necrosis Factor-Alfa, can perpetuate the inflammatory reaction, that can be modulated by the presence of the metal cerium. Conjugating together wound repairing, anti-microbial and immunomodulator actions, will make the dressing of the present invention an efficient therapeutic agent for burns and wounds of different etiologies, as: venous stases ulcers, pressure ulcers, plantar skin ulcers, complicated surgical wounds and burns.

The composition of the present invention has the appearance of an opaque gel and can be formulated as a dressing of varying sizes. The technique used for dressing preparation in the present invention consists of dissolving the cerium salt, particularly cerium nitrate, in a dermatologically acceptable carrier, preferably water, adding the solution of cerium salt to collagen and homogenize. In a preferred form, the mixture obtained must be homogenized to an alginate dispersion into an emollient, preferably propylene glycol, until it becomes a uniform mass. Then it is time to bottle it in specific forms according to desired formats and, subsequent freeze drying (lyophilization). After freeze drying (lyophilization) the product will have a spongy aspect, similar to fiber frame. The end product can then be sterilized through gamma radiation or ethylene oxide.

### DETAILED DESCRIPTION OF THE INVENTION

The present invention refers to a pharmaceutical composition for treating skin lesion comprising a cerium salt over a collagen matrix and a dermatologically acceptable carrier.

Preferably the cerium salt used is the cerium nitrate, in particular hexahydrated cerium nitrate, and the collagen used is bovine collagen type I.

Cerium salt can be present in the composition in amounts ranging from 0.1% to 5% by weight and the collagen varying from 5% to 95% by weight, based on the total weight of the composition. Preferably, cerium salt is present in amounts from 0.4% to 2.2% by weight and the collagen is present in amounts from 73% to 75% by weight, based on the total weight of the composition.

In a preferred embodiment of the invention, cerium salt is present in an amount of 75% by weight and the colagen is present in an amount of 0.4% by weight, based on the total weight of the composition.

In another preferred embodiment of the invention, cerium salt is present in an amount representing 73% by weight and collagen responds for 2.2% by weight, based on the total weight of the composition.

The composition of the present invention can also include a suspending agent. Preferably, this suspending agent will be an alginate, which can be chosen from sodium alginate and calcium alginate. The suspension agent can be present in amounts ranging from 1% to 20% by weight, based on the total weight of the composition, and its amount will preferably range from 5% a 15% by weight, particularly in an amount of 10% by weight.

The dermatologically acceptable carrier used in the composition of the present invention is preferably water.

The composition of the present invention can also be comprised of an emollient, preferably propylene glycol, which can be present in amounts varying from 1% to 20% by weight, based on the total weight of the composition, preferably in amounts ranging from 5% to 15% by weight, particularly in an amount of 10% by weight.

The composition of the present invention can be used in topical applications in a variety of lesion types, such as skin lesions involving the release of toxins related to microbial proteins on human or animal organisms, or those appointed as HSP; burns which involve burned skin toxin formation or LPC; chronically ulcerate skin lesions in which there is an overproduction of proteinase; skin lesions of difficult resolution, in which control of exudate overproduction is required; and critically infected or colonized skin lesions.

The present invention is also related to a dressing for treating skin lesion comprising the pharmaceutical composition of the present invention. Preferably, it is a lyophilized dressing and can be sterilized by gamma rays or ethylene oxide.

The present invention also refers to a cerium salt associated with a collagen in the preparation of a pharmaceutical composition or dressing according to the present invention.

In addition, the present invention also refers to a method for treating skin lesion by applying a pharmaceutical composition or a dressing as disclosed herein.

### Features of the Invention

Association of a substance promoting repair (collagen) to anti-microbial and immunomodulator substances (cerium salt, particularly cerium nitrate) in the form of a dressing.
Development of dressing formulation and production processes, using the tissue engineering technique, which will work as a cell proliferation matrix with hemostastatic, anti-microbial and immunomodulating properties, composed especially of collagen in association to a cerium salt, particularly cerium nitrate and optionally an (sodium or calcium) alginate.
   Advantages of the product in relation to the state of the technique.

The described dressing promotes tissue repair through the biological effects of collagen, which works as a structural support and facilitator for cell migration besides having a protective role of the newly-produced collagen in a collagenase-rich environment, common to chronic ulcers, in association with cerium salt antimicrobial and immunomodulating effects, described by severely burned patients and those suffering with chronic ulcers.

Preferably, a suspending agent should be used in the composition of the present invention. Best choice for the present invention suspending agent is an alginate. Particularly sodium or calcium alginate should be used. The presence of the (sodium or calcium) alginate works as a lesion humidity control mechanism, absorbing exudates excess, which avoids wound edge maceration and keeps the ideal humidity level in the wound.

The present invention dressing does not adhere to the bed, thus avoiding trauma during changes in the newly-formed tissue, and can be trimmed to the shape of the wound. In its range of applications there are indications for difficult resolution wounds, even those with critical bacteria colonization levels or those over which infection develops, through the work of the cerium metal antimicrobial action. Its clinical applications are, therefore: Burns, leg ulcers (venous stasis ulcers) artery and mixed ulcers, diabetic foot, pressure ulcers, surgical and trauma wounds.

Regarding the hydrocolloid dressings, the proposed product has the advantages of acting over protein and microbial toxins or HSP proteins and present antimicrobial, haemostatic properties, besides modulating lesions with proteases excess.

There are other collagen-containing dressings in the market, such as Fibracol®, Fibracol Plus®, Promogran®, amongst others. Such dressings have the limitation of not having an antimicrobial action, which can be critical in some clinical instances, as it is the case of chronic ulcers. These kind of ulcers are generally colonized by bacteria and it is known that critical levels of colonization are harmful to wound healing processes, even when devoid of infection. Products using only collagen, therefore, would have their application range widely limited to specific wound repair stages when no signal of infection or critical bacteria colonization could be detected and they should be replaced whenever these situations happen.

The present invention dressing offers cerium salt, particularly cerium nitrate, and its antimicrobial activity, as an association to collagen. The same substance is present in another antimicrobial and wound repair cream available in the market, Dermacerium®. Cerium salt, particularly cerium nitrate, brings additional property of immunomodulation, as already mentioned.

The choice made for cerium salt, particularly cerium nitrate, as an antimicrobial element present in the dressing was due, not only to its immunomodulating properties, as well as for the fact that there have been no relevant reports to date of the development of microbial resistance, even after years of use. Presentation in the form of dressings is more interesting than in the form of cream in the sense that it won't need so frequent replacements, and so will reduce costs and special personnel.

The market for dressings for different lesions is described in Table 1 below:

TABLE 1

| | World | | Compound |
|---|---|---|---|
| | Incidences (in | Healing Time | annual growth |
| Type of Lesion | million) | (days) | rate (CAGR) |
| Surgical Lesions | 97 | 14 | 3.1% |
| Trauma Lesions | 1.6 | 28 | 1.4% |
| Lacerations | 19.4 | 14 | 1.0% |
| Burns | 9.7 | 21 | 1.0% |
| Chronic Lesions | 26.3 | — | 7.4% |
| Carcinomas | 0.6 | 14 | 3.0% |
| Melanomas | 0.2 | 14 | 3.0% |
| Complex Skin Cancers | 0.2 | 28 | 3.0% |

Source: MedMarket Diligence, LLC

### Best Mode for Carrying Out the Invention

Detailed illustrative embodiments of the invention disclosed herein exemplify the invention and are currently considered to be the best embodiments for such purposes. They are provided by way of illustration and not limitation of the invention. Various modifications thereof will occur to those skilled in the art, and such modifications are within the scope of the claims which define the present invention.

The best modes for carrying out the present invention are:

a) A pharmaceutical composition for treating skin lesion comprising from 0.4% to 2.2% by weight of a cerium salt over a matrix comprising from 73% to 75% by weight of bovine collagen type I and water, said composition optionally comprising calcium or sodium alginate as a suspending agent and 10% by weight of propylene glycol as emollient.

b) A dressing for treating skin lesion comprising a pharmaceutical composition comprising from 0.4% to 2.2% by weight of a cerium salt over a matrix comprising from 73% to 75% by weight of bovine collagen type I and water, said composition optionally comprising calcium or sodium alginate as a suspending agent and 10% by weight of propylene glycol as emollient.

c) A method for treating skin lesion comprising the step of applying a pharmaceutical composition or a dressing as disclosed above on said skin lesion.

### Sample Formulations

### Formulation 1:

| | Preferred | Special | |
|---|---|---|---|
| | Concentration | Concentration | |
| Raw material | (% w/w) | (% w/w) | Properties |
| Bovine Collagen | 5 to 95% | 75% | Hemostatic, |
| Type I | | | chemotactic and |
| | | | matrix for cell |
| | | | migration |
| Cerium Nitrate | 0.1% to 5% | 0.4% | Antimicrobian, |
| | | | immunomodulador, |
| | | | anti-inflammatory |
| | | | (reduction of TNF-α |
| | | | levels). |
| Propylene Glycol | 1% to 20% | 10% | Emollient |

| | | |
|---|---|---|
| Water | q.s.p. 100% | Carrier |

### Formulation 2:

| | Preferred | Special | |
|---|---|---|---|
| | Concentration | Concentration | |
| Raw material | (% w/w) | (% w/w) | Properties |
| Bovine Collagen | 5% to 95% | 73% | Hemostatic, |
| Type I | | | chemotactic and |
| | | | matrix for cell |
| | | | migration |
| Cerium Nitrate | 0.1% to 5% | 2.2% | Antimicrobian, |
| | | | immunomodulador, |
| | | | anti-inflammatory |
| | | | (reduction of TNF-α |
| | | | levels). |
| Propylene Glycol | 1% to 20% | 10% | Emollient |

| | | |
|---|---|---|
| Water | q.s.p. 100% | Carrier |

### Formulation 3:

| | Preferred | Special | |
|---|---|---|---|
| | Concentration | Concentration | |
| Raw material | (% w/w) | (% w/w) | Properties |
| Bovine Collagen | 5 to 95% | 75% | Hemostatic, |
| Type I | | | chemotactic and |
| | | | matrix for cell |
| | | | migration |
| Cerium Nitrate | 0.1% to 5% | 0.4% | Antimicrobian, |
| | | | immunomodulador, |
| | | | anti-inflammatory |
| | | | (reduction of TNF-α |
| | | | levels). |
| Sodium alginate | 1% to 20% | 10% | Suspending agent, |
| | | | exudate absorber |
| Propylene Glycol | 1% to 20% | 10% | Emollient |

| | | |
|---|---|---|
| Water | q.s.p. 100% | Carrier |

### Formulation 4:

| | Preferred | Special | |
|---|---|---|---|
| | Concentration | Concentration | |
| Raw material | (% w/w) | (% w/w) | Properties |
| Bovine Collagen | 5% to 95% | 75% | Hemostatic, |
| Type I | | | chemotactic and |
| | | | matrix for cell |
| | | | migration |
| Cerium Nitrate | 0.1% to 5% | 0.4% | Antimicrobian, |
| | | | immunomodulador, |
| | | | anti-inflammatory |
| | | | (reduction of TNF-α |
| | | | levels). |
| Calcium alginate | 1% to 20% | 10% | Suspending agent, |
| | | | exudate absorber |
| Propylene Glycol | 1% to 20% | 10% | Emollient |

| | | |
|---|---|---|
| Water | q.s.p. 100% | Carrier |

### Formulation 5:

| | Preferred | Special | |
|---|---|---|---|
| | Concentration | concentration | |
| Raw material | (% w/w) | (% w/w) | Properties |
| Bovine collagen | 5 to 95% | 73% | Hemostatic, |
| type I | | | chemotactic and |
| | | | matrix for cell |
| | | | migration |
| Cerium Nitrate | 0.1% to 5% | 2.2% | Antimicrobian, |
| | | | immunomodulador, |
| | | | anti-inflammatory |
| | | | (reduction of TNF-α |
| | | | levels). |
| Sodium alginate | 1% to 20% | 10% | Suspending agent, |
| | | | exudate absorber |
| Propylene Glycol | 1% to 20% | 10% | Emollient |

| | | |
|---|---|---|
| Water | q.s.p. 100% | Carrier |

### Formulation 6:

| | Preferred | Special | |
|---|---|---|---|
| | Concentration | concentration | |
| Raw material | (% w/w) | (% w/w) | Properties |
| Bovine collagen | 5% to 95% | 73% | Hemostatic, |
| type I | | | chemotactic and |
| | | | matrix for cell |
| | | | migration |
| Cerium Nitrate | 0.1% to 5% | 2.2% | Antimicrobian, |
| | | | immunomodulador, |
| | | | anti-inflammatory |
| | | | (reduction of TNF-α |
| | | | levels). |
| Calcium alginate | 1% to 20% | 10% | Suspending agent, |
| | | | exudate absorber |
| Propylene Glycol | 1% to 20% | 10% | Emollient |

| | | |
|---|---|---|
| Water | q.s.p. 100% | Carrier |

The examples shown above are preferred and illustrative variations of the present invention composition and should not be interpreted as limitations to it. In this regard, it should be understood that the scope of the present invention comprehends the possibility of other variations to the composition, these being limited only by the context of the claims here incorporated, with possible equivalents hereon included.

### Bacteriostatic Evaluation Test

Bacteriostatic evaluation of the present invention product was performed according to the “Manual de Saneantes do Instituto Nacional de Controle de Qualidade em Saúde—Item 8-B: Métodos para Avaliação da Atividade Inibitória de Preparações Líquida, Cremosa e Sólida—Método da Placa de Ágar”—January 1992, for the following microorganisms: Staphylococcus aureus ATCC 6538 and Salmonella choleraesuis ATCC 10708. Test result proved the bacteriostatic action of the product after a clear inhibition zone formed around the sample.

1. Pharmaceutical composition for treating skin lesion, characterized by comprising a cerium salt over a collagen matrix and a dermatologically acceptable carrier.

2. Pharmaceutical composition according to claim 1, characterized in that the cerium salt is cerium nitrate, preferably hexahydrated cerium nitrate.

3. Pharmaceutical composition according to claim 1, characterized in that the collagen used is bovine collagen type I.

4. Pharmaceutical composition according to claim 1, characterized in that the cerium salt is present in an amount ranging from 0.1% to 5% by weight, and the collagen is present in an amount ranging from 5% to 95% by weight, based on the total weight of the composition.

5. Pharmaceutical composition according to claim 4, characterized in that the cerium salt is present in an amount of 0.4% by weight and the collagen is present in an amount of 75% by weight.

6. Pharmaceutical composition according to claim 4, characterized in that the cerium salt is present in an amount of 2.2% by weight and the collagen is present in an amount of 73% by weight.

7. Pharmaceutical composition according to claim 1, characterized in that it further comprises a suspending agent.

8. Pharmaceutical composition according to claim 7, characterized in that the suspending agent is an alginate, selected from the group consisting of sodium alginate and calcium alginate.

9. Pharmaceutical composition according to claim 7, characterized in that the suspending agent is present in an amount ranging from 1% to 20% by weight, based on the total weight of the composition.

10. Pharmaceutical composition according to claim 1, characterized in that the dermatologically acceptable carrier is water.

11. Pharmaceutical composition according to claim 1, characterized in that it further comprises an emollient.

12. Pharmaceutical composition according to claim 11, characterized in that the emollient is propylene glycol.

13. Pharmaceutical composition according to claim 11, characterized in that the emollient is present in an amount ranging from 1% to 20% by weight, based on the total weight of the composition.

14. Pharmaceutical composition according to claim 1, characterized in that the it is designed for topical application in skin lesions involving the release, in human or animal organisms, of toxin related to microbial proteins or those denominated as HSP, in burns involving the formation of burned skin toxin or LPC, in chronic ulcerated skin lesions in which there is proteinase overproduction, and in skin lesions which were critically infected or colonized.

15. Pharmaceutical composition according to claim 7, characterized in that it is designed for topical application in skin lesions of difficult resolution, in which control of exudate overproduction is required.

16. Pharmaceutical composition according to claim 1, characterized in that it is designed for topical application in lesions selected from venous stasis ulcers, pressure ulcers, perforating plantar wounds and complex surgical wounds and burns.

17. Dressing for treating skin lesion, characterized by comprising a pharmaceutical composition as defined in claim 1.

18. Dressing according claim 17, characterized in that it is lyophilized.

19. Dressing according to claim 17, characterized in that it is sterilized by gamma radiation or ethylene oxide.

20. Use of cerium salt in association with collagen, characterized in that it is for preparing a pharmaceutical composition as defined in claim 1.

21. Method for treating skin lesion, comprising applying a pharmaceutical composition as defined in claim 1 to the skin lesion.
